(19) United States
(12) Patent Application Publication
(10) Pub. No.: US 2011/0136101 A1
(43) Pub. Date: Jun. 9, 2011

(54) Immortalized Human Fetal Liver Cells

(76) Inventors: Jan Holgersson, Vastra Frolunda (SE); Suchitra Holgersson, Vastra Frolunda (SE); Setara Begum, Huddinge (SE); Marika I. Kleman, Alvsjo (SE); Monica Ek, Solna (SE)

(21) Appl. No.: 12/960,748

(22) Filed: Dec. 6, 2010

Related U.S. Application Data
(60) Provisional application No. 61/266,822, filed on Dec. 4, 2009.

(51) Int. Cl. C12Q 1/70; C12N 5/071; C12Q 1/02

(52) U.S. Cl. 435/5; 435/370; 435/29

(57) ABSTRACT

The present invention provides immortalized fetal liver cells that express the oncogene Simian virus (SV 40) large T-antigen and specific hepatocyte markers and a method for producing same.

### RELATED APPLICATIONS

This application claims the benefit of U.S. Provisional Application U.S. Ser. No. 61/266,822, filed Dec. 4, 2009 the contents of which is hereby oncorporated by reference in its entireties.

### FIELD OF THE INVENTION

The present invention relates to immortalized human fetal hepatocytes.

### BACKGROUND OF THE INVENTION

Cultured human fetal liver (HFL) cells are unique in their potential utility in a number of diagnostic and clinical applications. Even though they have an extended life-span compared to adult primary cells, they eventually lose their ability to divide and enter senescence after a finite number of population doublings. For applications such as the bioartificial liver support requiring large numbers of cells, or applications requiring cells of reproducible quality such as in the case of drug toxicity testing, a renewable source of cells that is constant and can be expanded into large numbers is necessary. Primary cultures from explanted animal or human tissue do not fill this need (1-3).

Immortalized cells, that are free from cell aging and have acquired ability for infinite proliferation in the course of subculturings of the primary culture, have stable and equal properties. Still, many of such immortalized cells lose part or whole of morphology or function that the cells originally possessed in the organisms, and therefore in the experiments using such immortalized cells, it has been considered difficult to precisely reflect their original properties that the cells displayed in their deriving tissues.

Thus a need exists for an immortalized human fetal liver cell that retains similar morphological and function charatceristics to that of the primary cells.

### SUMMARY OF THE INVENTION

The present invention provides an immortalized human fetal liver cell line containing actively expressing SV40 LT genes. The immortalized cell line maintains the phenotypic properties of human fetal liver cells and is capable of differentiating in vivo into a hepatocyte, or a cholangiocyte. Also provided is an immortalized in vitro cell culture where the cells are derived from fetal liver tissue of a human. The cells in the culture express express albumin, cytokeratin 8 (CK8), cytokeratin 18 (CK18), cytokeratin 19 (CK19), cytochrome P450 2A4/7 isoenzyme, HNF4α, and glucose-6-phosphatas; do not express cancer associated markers such as, p-53, MOC-31 or Ber EP4; and are CD271⁻, CD34⁻ and CD35⁻. The cells are capable of proliferating in a culture and differentiate in vivo into a hepatocyte, or a cholangiocyte. The cells are capable of storing glycogen. The cells do not form tumors in vivo.

Compounds which effect proliferation, differentiation or survival of liver cells are identified contacting the immortalized fetal liver cells with a test compound and determining if the compound has an effect on proliferation, differentiation or survival of the cells. Similarly, the metabolite of a test compound is determined. Metabolites are identified by screening the culture medium after contacting the immortalized fetal liver cells with the test compound. Metabolites are identified by methods know in the art such a HPLC, Mass spectroscopy or gel electrophoresis. Anti-viral activity of a test compound is determined by introducing a virus to an immortalized fetal liver cell in the presence or absence of a test compound and determining the survival rate of the cells. An increase in survival rate in the presence of the test compound compared to the absence of the test compound indicates that the test compound has anti-viral activity. Infectivity of a virus is determined by contacting an immortalized fetal liver cell with a virus and determining the effect of proliferation or survival of the cells. A decrease of survival or proliferation as compared to a cell culture that has not been contacted with the virus indicates the virus can infect liver cells. In contrast, a similarity of survival or proliferation as compared to a cell culture that has not been contacted with the virus indicates the virus does not infect liver cells.

Proliferation and or survival is determined by methods know in the art such as BrdU assay. Differentiation is determined morphologically or histologically by determining hepatic cell surface markers. The virus is a liver trophic virus such as hepatitis virus A, hepatitis virus B, or hepatitis virus C.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. Phase contrast microscopy of SV40LT-HFL cells: (A) one day after transfection, (B) derived from one colony after 13 days of culture in medium with puromycin, (C) from a second colony from the same plate, (D) from a single colony after 2 months with puromycin selection, and (E & F) showing cells with two or three nuclei, typical of differentiating and mature hepatocytes. Magnification A-E 20×; F 40×.

FIG. 2. Immunofluorescent staining of different bulk clones of SV40LT-HFL cells (bulk clones 4A, 1A, 1B and 3A) in passages 2-20 showing from left to right the detection of cytoplasmic staining of the hepatic markers CK8 and 18 and nuclear staining of the transcriptional factor HNF4α (in green). Cells are counterstained with the nuclear stain DAPI (blue color). Representative staining of SV40LT-HFL cells with isotype negative controls mouse IgG1 and IgG2a is shown in the upper panel to the right. Magnification: ×20.

FIG. 3. Immunofluorescent staining with an antibody to SV40 LT antigen demonstrated strong expression of the SV40 LT antigen in nuclei of all the cells (bulk clone 4A) in (A) passage 10 and (B) passage 20. Nuclear staining was with DAPI. (C) Immunofluorescent staining of SV40LT-HFL 4A cells in passage 11 and the liver cancer cell line, HepG2, for cancer-associated markers Ber EP4, MOC-31 and p53. The SV40LT-HFL 4A cells were negative for all the three markers, while Hep G2 cells were positive for Ber EP4 and MOC-31, but not p53. Magnification: ×20. (D) Histochemical staining of the transfected SV40LT-HFL 4A cells and liver cancer cell line HepG2 cells showing the expression of glucose-6-phosphatase and glycogen storage in the transfected cells but not the cancer cell line Hep G2.

FIG. 4. (A) Flow cytometry of SV40LT-HFL 4A cells showed expression of the stem cell markers EPCAM, CD133, CD90 and Dlk-1in early passages (p11-12), which was decreased or absent in a later passage (p22). In addition, the cells also expressed the hepatic markers CK 8 and CK18 confirming our immunocytochemical results. No expression of CD271, a mesenchymal stem cell marker, was observed. The cells did not express the hematopoietic marker CD34 or the leukocyte marker CD45. (B) RT-PCR analysis of SV40LT-HFL 4A cells in passage 10 showing expression of detoxifying factors CYP3A4, CYP3A7, albumin and transcription factor HNF-4α. β-actin was used as housekeeping gene. HepG2 cells served as positive control, and expressed CYP3A4, CYP3A7, HNF-4-α, HNF-1β and albumin transcripts as revealed by RT-PCR.

FIGS. 5&6. Expression of human liver-specific markers in the livers of nude mice transplanted with SV40 LT-HFL cells. One million SV40 LT-HFL 4A cells were transplanted into the spleens of retrorsine-treated nude mice that underwent 40% partial hepatectomy at the time of transplantation. Immunohistochemistry was performed on fresh frozen liver sections of transplanted animals, and small clusters of human CK8-, CK19-, hepatocyte specific antigen-, α-fetoprotein-, and c-Met-expressing, engrafted cells (dark brown) with hepatocyte morphology were detected throughout the liver. Biopsy sections from patients with liver cancer served as positive control and staining of liver sections from sham transplanted animals served as negative control. HE was used as counter-stain. Magnification: ×40. CK, cytokeratin; Hep Ag, hepatocyte-specific antigen; AFP, α-fetoprotein.

### DETAILED DESCRIPTION OF THE INVENTION

The present invention provides an immortalized human fetal hepatocyte (iHuFH) cell line that expresses the oncogene Simian virus (SV 40) large T-antigen and specific hepatic markers and a method for producing same. The iHuFH cells retain all the hepatic markers and hepatic transcription factors over several passages.

Introduction of SV40 into primary cultures of human cells with either whole virus or plasmid DNA results in enhanced cell division and prolonged life span, and in a few cases will lead to the development of an immortalized cell line (17). In this study the successful establishment of a human fetal liver cell line by introduction of SV40 large T antigen into normal primary human fetal liver (HFL) cells is described. The newly established immortalized fetal hepatocytes (referred to herein sas iHuFH) revealed morphologic characteristics of primary hepatocytes in standard culture systems and expressed many liver enriched markers, such as albumin, HNF-4α, and CYP3A4/7. Immunohistochemistry assay demonstrated that the cells expressed liver-specific markers such as glucose-6-phosphatase and glycogen. Importantly, using cancer-associated markers such as these Ber-EP4, MOC-31 or p53; it was demonstrated that the transfected cells in vitro did not express any of these markers. The markers, Ber-EP4 and MOC-31 have been demonstrated to be expressed on a wide range of normal and neoplastic human tissues, except hepatocytes, parietal cells, and apical cell layers in squamous epithelia (18, 19). The oncogenic potential of the SV40 large T antigen resides in part in its ability to bind and inactivate many of the activities of the tumor suppressor p53 (4-6). However, the SV40 LT Ag transfected cells of the invention did not stain positive for p53. Furthermore, transplanted cells in vivo did not demonstrate tumor formation at 4 weeks after transplantation, or expression of p53. Thus, the present invention provides an in vitro expandable fetal liver progenitor cells by means of immortalization and without inducing a transformed phenotype and disrupting their differentiation potential. The cell line of the invention would facilitate studies on cell engraftment and differentiation within the hepatic parenchyma.

Development of cell therapy-based strategies for the treatment of liver failures and of inherited metabolic diseases has become a necessity because of the limitations of orthotopic liver transplantation, including shortage of donor livers. This shortage limits also the availability for hepatocytes, terminally differentiated cells that cannot be expanded in vitro. Thus, other alternative sources of hepatocytes, such as hepatic stem cells, have to be explored. Fetal hepatic cells have certain intrinsic properties compared to adult hepatocytes which make it advantageous to use these cells for establishment of immortalized cell lines. For cell therapy applications, a temporally controlled expression of the immortalizing transgene would permit reversion of the immortalized phenotype prior to cell transplantation. In most cases, murine hepatic stem cell lines have been used to study immortalization and transformation in vitro (20, 21). However, characteristics of murine stem cells cannot be extrapolated to their human counterparts, therefore it is important to establish human hepatic stem/progenitor cell lines to study the molecular events involved in their proliferation and differentiation in vitro as well as their fate in vivo after transplantation. Thus, the present provides new approaches to expand hepatic progenitor cells, analyze their fate in animal models aiming at cell therapy of hepatic diseases.

As the SV40 gene is integrated randomly into the host genome (6), it is possible that the phenotypic characteristics and endogenous functions of the transfected cells could be different from those of parental cells. Thus the phenotypic and functional characteristics of SV40LT-HFL cells and normal HFL cells were compared. The data showed that transfected HFL cells (i.e. iHuFHs) stably maintained the morphologic and phenotypic features of normal HFL cells, had an extended life span and could be propagated in large numbers. These cells expressed detectable levels of cytokeratin 8/18, which are established hepatic markers. Moreover, the in vitro and in vivo data revealed that the transfected cells had the capacity to differentiate into both hepatocytes as well as cholangiocytes indicating their bipotentiality. In the developing liver, both hepatocytes and cholangiocytes are known to differentiate from a common cell precursor (23). Suzuki et al (24) have enriched and isolated bipotent hepatic progenitors from rodents. However, because cells from different species are intrinsically different, our in vivo data using human hepatic precursors is more reliable and appropriate to establish preclinical procedures of hepatic cell transplantation.

Flow cytometric analysis showed that in the early passages, these cells were positive for the hepatic stem cell markers, (25) EPCAM, CD133, CD90 and Dlk-1, but not CD34 or CD45 indicating the non-hematopoietic origin of these cells.

RT-PCR and immunocytochemical assays demonstrated that, the transfected cells expressed the genes and proteins (positive cytoplasmic staining for CY3A/7) and the transcription factor HNF 4α. HNF-4α is required for the PXR and CAR-mediated transcriptional activation of CYP3A4 and is a transcription factor that is involved in the regulation of the expression of several liver specific genes. CYP3A4 is believed to be the predominant cytochrome P450s expressed in adult human liver and is involved in the oxidation of the largest range of substrates of all the CYPs. CYPs are the major enzymes involved in drug metabolism and bioactivation. Therefore, these immortalized SV40LT-HLF cells may be useful for the development of diagnostic tools for toxicity studies.

In conclusion, the inventors have established a SV40LT-HFL cell line which preserved the characteristics of normal HFL cells differentiated in culture, had an increased growth capacity and so far has retained a stable phenotype up to passage 26. The present study demonstrates that conditionally immortalized fetal hepatocytes are a potential cell source for liver based transplantation and could potentially overcome the limited lifespan and inability to proliferate in vitro primary adult hepatocytes. Therefore, the immortalized SV40LT-HFL cell line may be useful for the development of diagnostic strategies, especially in toxicology and bioartificial liver support system to treat liver failure.

“Oncogene” as used herein is intended to mean a gene whose action promotes cell proliferation. Oncogenes are altered forms of proto-oncogenes and are often expressed in cancerous cells. Exemplary oncogenes include large T antigen, myc, abl, ras, and raf.

A “SV40 Large T Antigen” (SV-40 LTA) oncogene is intended to encompass any nucleotide sequence which encodes a protein having the function of polyoma (or SV-40) LTa and which is capable of being expressed in the host cell, e.g., a hepatocyte.

“Immortalized cell” is a cell that have been modified to undergo indefinite numbers of successive passage”

“Immortalized cell line” as used herein means a cell line that can replicate and be maintained indefinitely in in vitro cultures under conditions that promote growth, preferably at least over a period of a year or years.

Immortalization” as used herein refers to a process which increases the lifespan of a cell, particularly a primary cell, so that the resulting cell line is capable of being passaged many more times than the primary cell is capable of being passaged.

“Cell line” as used herein is a population or mixture of cells of common origin growing together after several passages in vitro. By growing together in the same medium and culture conditions, the cells of the cell line share the characteristics of generally similar growth rates, temperature, gas phase, nutritional and surface requirements. The cell line can become more homogenous with successive passages and selection for specific traits. Clonal cells are those which are descended from a single cell. A cloned cell culture is a cell culture derived from a single cell.

“Isolated cell” refers generally to a cell that is not associated with one or more cells or one or more cellular components with which the cell is associated in vivo. For example, an isolated cell may have been removed from its native environment.

“In vitro” as used herein denotes outside, or external to, animal or human body. The term “in vitro” as used herein should be understood to include “ex vivo”. The term “ex vivo” typically refers to tissues or cells removed from an animal or human body and maintained or propagated outside the body, e.g., in a culture vessel from propagation, e.g., ex vivo propagation, of a cell that has been removed from its native environment.

“Cell population” refers generally to a grouping of cells. Unless indicated otherwise, the term refers to a cell grouping consisting of or comprising isolated cells as defined herein.

A cell population may consist of cells having a common phenotype or may comprise at least a fraction of cells having a common phenotype. Cells are said to have a common phenotype when they are substantially similar or identical in one or more demonstrable characteristics, including but not limited to morphological appearance, the presence, absence or level of expression of particular cellular components or products, e.g., RNA, proteins or other substances, activity of certain biochemical pathways, proliferation capacity and/or kinetics, differentiation potential and/or response to differentiation signals or behaviour during in vitro cultivation (e.g., adherence, non-adherence, monolayer growth, proliferation kinetics, or the like). Such demonstrable characteristics may therefore define a cell population or a fraction thereof.

When a cell population is said herein to be “heterogeneous”, this generally denotes a cell population comprising two or more cells or fractions of cells not having a common phenotype, e.g., a cell population comprising cells of two or more different cell types. By means of example and not limitation, a heterogeneous cell population can be isolated from liver, and may comprise diverse liver cell types, including but not limited to hepatocytes (e.g., large and small hepatocytes), cholangiocytes, Kupffer cells, hepatic stellate cells (Ito cells) and liver endothelial cells.

When a cell population is said herein to be “homogeneous”, it consists of cells having a common phenotype. A cell population said herein to be “substantially homogeneous” comprises a substantial majority of cells having a common phenotype. A “substantially homogeneous” cell population may comprise at least 70%, e.g., at least 80%, preferably at least 90%, e.g., at least 95%, or even at least 99% of cells having a common phenotype, such as the phenotype specifically referred to (e.g., the phenotype of progenitor cell or stem cell). As used herein, the term “substantially homogeneous” as used herein may thus also encompass a homogeneous population

The term “cell population comprising a progenitor or stem cell” refers to a cell population as defined herein comprising at least one progenitor or stem cell and typically a fraction of progenitor cells or stem cells, as defined herein. Usually, the progenitor or stem cells of the said fraction may have a common phenotype.

A “selectable marker” is a genetic determinant which makes it possible to provide culture conditions which favour the growth of cells possessing the marker, compared to cells which do not. An antibiotic resistance gene is an example of a selectable marker.

The terms “differentiation”, “differentiating” or derivatives thereof as used herein denote the process by which an unspecialised or a relatively less specialised cell becomes relatively more specialised. In the context of cell ontogeny, the adjective “differentiated” is a relative term. Hence, a “differentiated cell” is a cell that has progressed further down a certain developmental pathway than the cell it is being compared with. A differentiated cell may, for example, be a terminally differentiated cell, i.e., a fully specialised cell that takes up specialised functions in various tissues and organs of an organism, and which may but need not be post-mitotic. In another example, a differentiated cell may also be a progenitor cell within a differentiation lineage, which can further proliferate and/or differentiate. Similarly, a cell is “relatively more specialised” if it has progressed further down a certain developmental pathway than the cell it is being compared with, wherein the latter is therefore considered “unspecialised” or “relatively less specialised”. A relatively more specialised cell may differ from the unspecialised or relatively less specialised cell in one or more demonstrable phenotypic characteristics, such as, for example, the presence, absence or level of expression of particular cellular components or products, e.g., RNA, proteins or other substances, activity of certain biochemical pathways, morphological appearance, proliferation capacity and/or kinetics, differentiation potential and/or response to differentiation signals, etc., wherein such characteristics signify the progression of the relatively more specialised cell further along the said developmental pathway. Non-limiting examples of differentiation may include, e.g., the change of a pluripotent stem cell into a given type of multipotent progenitor or stem cell, the change of a multipotent progenitor or stem cell into a given type of unipotent progenitor or stem cell, or the change of a unipotent progenitor or stem cell to more specialised cell types or to terminally specialised cells within a given cell lineage. Differentiation of an unspecialised or less specialised cell to a more specialised cell may proceed through appearance of cells with an intermediate degree of specialisation.

Any virus may be used to transform the cells which contains a transforming nucleic acid sequence, and can extend the lifespan of the cells. Such viruses include papilloma viruses, SV40 viruses, and hybrid viruses containing SV40 transforming genes, such as Adenol2-SV40. These viruses express SV40 T antigen, which is a multi-functional transforming protein that binds p53 and Rb gene product, but any molecule that binds to p53 theoretically could be employed to achieve the same end. T antigen contains a domain that has been identified in adenovirus E1A.

Alternatively, plasmids may be used to transfect the cells with nucleic acid capable of transforming the cells. Such plasmids may include sequences from SV40 virus, such as RSV-T (pRSV-I), sequences from papilloma viruses, or any other transforming sequences.

SV40-based viruses are preferable for carrying out the present invention. The SV40 virus and methods for obtaining the virus and viral DNA have been described in the literature. See, for example, Rhim et al, 1981, Proc. Natl. Acad. Sci., Vol. 78, pp. 313-317; Rhim, Anticancer Research, 1989, Vol. 9, pp. 1345-1366; and Rhim et al, Science, 1985, Vol. 227, pp. 1250-1252.

SV40-based plasmids are also preferable for use in the present invention. Particularly preferable is the plasmid pRSV-T, the use of which is described in Reddel et al, 1988, Cancer Research, Vol. 48, pp. 1904-1909. This plasmid contains the SV40 early region genes and the Rous Sarcoma Virus long terminal repeat.

Infection of the cells by the SV40 virus, or transfection of the cells with a plasmid containing SV40 sequences can be confirmed by assaying for the presence of SV40 antigens. Such assays may include the use of polyclonal or monoclonal antibodies to SV40 antigens.

The cell lines can be tested to assure that they retain the phenotypic traits of human fetal hepatocytes. Such characteristics which can be tested include, for example, morphology, saturation density, population doubling time, karyotype and specific cytokeratin synthesis. Morphological studies can be conducted using methods of microscopy such as phase-contrast and brightfield microscopy. Saturation density and population doubling time can be analyzed by releasing cells from a tissue culture surface, and counting cells by any means, particularly by the use of a Coulter counter.

The cell lines of the invention are useful in a variety of methods. Such methods include a model system for the human liver. Such a model is useful for toxicity testing. For toxological applications of the cell lines, see, A Critical Evaluation of Alternatives to Acute Ocular Irritation Testing, 1987, Frazier et al, eds., Mary Ann Liebert, Inc., New York, N.Y. Furthermore, the cell lines can be utilized as model systems to experiment liver regerationa and bioartifical liver support system, and as a model system for viral infection and diseases of the liver.

### EXAMPLES

### Example 1

### General Method

Human Fetal Liver Cells Preparation and Culture

Permission for the present study was granted from the local ethical committee. Primary human fetal liver cells were collected from a legally aborted human fetus 6.5 weeks of gestational age. The female donating the fetal tissue was serologically screened for syphilis, toxoplasmosis, rubella, HIV-1, cytomegalovirus, hepatitis B and C, parovirus and herpes simplex types 1 and 2 and found negative. A single cell suspension was prepared as described earlier (14). Cells were inoculated in a collagen-coated 75 cm² tissue culture flask. The culture medium consisted of Dulbecco's Modified Eagle medium (DMEM, Gibco BRL, Grand Island, N.Y.) supplemented with 10% inactivated human AB serum, VEGF (5 ng/mL), IL-6 (2 ng/ml), HGF (30 ng/ml; Biosource, C A, USA), EGF (20 ng/ml; Millipore, Solna, Sweden), FGF (10 ng/ml; Cambrex, N.J. USA), 5% (v/v) of non-essential amino acids (NEAA), 5% (v/v) sodium pyruvate and 5% (v/v) L-glutamine (Invitrogen, Taastrup, Denmark). Primary cultures lasted for 20 days with medium changes every third day. All cultures were maintained at 37° C. in a humidified atmosphere of 5% CO₂.

Construction of the CMV/SV40LT/PAC Plasmid

The SV40 large T antigen cDNA was amplified by PCR from a plasmid containing its full length sequence using 5′-cgc ggg ctc gag acc atg gat aaa gtt tta aac-3′ and 5′-cgc ggg gcg gcc get tta tgt ttc agg ttc agg-3′ as forward and reverse primers, respectively. The vector used to generate stable transfectants were bidirectional having the Spleen focus-forming virus (Sffv) long terminal repeat (Ltr) upstream of a polylinker, a splice donor and acceptor site, and the bidirectional poly(A) addition signal of SV40; opposite in orientation to this transcription unit, and utilizing the poly(A) signals from the opposite direction was a second transcription unit consisting of the HSV TK promoter followed by the coding sequences for puromycin acetyltransferase (Sffv/PAC; N. Chiu, J. Holgersson and B. Seed, unpublished). The SV40LT cDNAs was swapped into the Sffv/PAC vector using Xho I and Not I. Thereafter, the Sffv Ltr was removed and the IE CMV promoter from CDM8 cloned into the vector using Spe I and Xho I, thus creating CMV/SV40LT/PAC.

Transfection

Cells were plated one day before transfection in collagen-coated 6-well plates (BD, San Jose, Calif., USA) to reach a confluency of 80% at time of the transfection. The Avr II linearized SV40LT plasmid mixed with Plus reagent (Invitrogen Ltd, Paisley, UK) in Opti-MEM (Gibco, Paisley, UK) was incubated for 30 min at room temperature with Lipofectamine LTX (Invitrogen Ltd, Paisley, UK) according to the manufacturer's instructions before adding the mixture to the cells. The medium was changed from Opti-MEM to the ordinary growth medium after 6 hrs. Cells were split 1: 12 one day after transfection and selection medium containing puromycin was added the following day. Bulk-selected cells growing in the presence of puromycin were checked for SV40 large T antigen expression by immunocytochemistry (see below).

Growth Assay

Stable Transfected SV40 LT-HFL cells cultured for 2.5 months in passage 9 were seeded in collagen-coated 6-well plates (BD Biosciences, N.J., USA) at a density of 80,000 cells/well and the growth was followed for 7 days. Media of cells was changed every third day. At 24-h intervals, cells were detached, spun at 200 g for 5 min and cell numbers in triplicates were determined by a manual haemocytometer. Population doubling time (PDT) was calculated at the time of exponential growth (log phase), i.e. between 48 h and 96 h after initial plating,

Detection of Hepatic Markers

Hepatic markers expressed by the transfected SV40LT-HFL cells were determined in early and late passages using three different assays: a) immunocytochemical analysis, b) RT-PCR and c) flow cytometry.

Immunocytochemical Analysis.

Immunofluorescence analysis of the SV40LT-HFLcells was done in their early generation time as well as later passages. Transfected cells were inoculated on to collagen-coated glass slides (BD Biosciences, N.J., USA) and allowed to adhere for 1 day in DMEM (Invitrogen, CA, USA) with 10% inactivated human AB serum. Adherent cells were washed twice with PBS and fixed in 4% paraformaldehyde for 10 minutes at room temperature, washed twice with PBS and blocked in 3% BSA/PBS. The cells were incubated overnight at 4° C. with antibodies specific for human CK8 (Santa Cruz Biotechnology, Santa Cruz, Calif., USA) diluted 1:100 in 3% BSA/PBS, human CK18 (Morphosys AbD GmbH, Dusseldorf, Germany) diluted 1:50, human CK19 (Morphosys AbD GmbH, Düsseldorf, Germany) diluted 1:100, human albumin (Sigma-Aldrich, St. Louis, Mo., USA) diluted 1:100, human CYP3A4 (Santa Cruz Biotechnology, Inc.Heidelberg, Germany) diluted 1:100, human HNF-1β (Santa Cruz Biotechnology, Inc.Heidelberg, Germany) diluted 1:100, and the SV40 large T antigen (Santa Cruz Biotechnology, Inc.Heidelberg, Germany) diluted 1:100. Antibodies to detect cancer associated markers such as anti-BER EP4 (an epithelial membrane antigen) diluted 1:100, anti-MOC 31 diluted 1:100 and anti-p53 (a tumor suppressor marker) were also used. The hepatoma cell line Hep G2 was used as positive control cells. As isotype controls mouse IgG1 (Dako, Glostrup, Denmark) and mouse IgG2a (Sigma-Aldrich, St Louis, Mo., USA) were used in dilutions that equal the IgG concentrations of the primary antibodies. After washing two times with PBS, cells were incubated with secondary antibodies (Alexa Fluor 488 rabbit anti-mouse IgG; Invitrogen Ltd., Eugene, Oreg., USA) for 1 hour and counterstained with 4′,6-diamidino-2-phenylindole (DAPI) for 10 minutes. Antibody binding was detected in an Axiovert 40CFL fluorescence microscope (Carl Zeiss Microimaging Gmbh, Gottingen, Germany) and pictures captured using the Axiocam MRm digital camera from Carl Zeiss and the Axiovision software (Carl Zeiss Microimaging Gmbh). Liver specific markers such as glucose-6-phosphatase (G-6-P) and glycogen were demonstrated in transfected cells as described earlier (15, 16).

RT-PCR.

Total RNA was isolated from transfected cells using RNA-Bee™ (Tel-Test, Inc. Friendswood, Tex., USA). The medium was aspirated, the cells washed with PBS and 1 ml of RNA-Bee™ added directly to each flask and the cells solubilized. Thereafter the RNA was extracted according to the manufacturer's instructions. The RNA concentration was determined by UV absorbance at 260 nm using a DU 730 spectrophotometer (Beckman Coulter, Fullerton, Calif., USA). RNA was collected once from cells in their 10^{th} passage and again in their 20^{th} passage. One μg of total RNA was reverse transcribed into cDNA at 50° C. using SuperScript™ first-strand synthesis kit (Invitrogen, Carlsbad, Calif., USA). The cDNA samples were subjected to PCR amplification using primers specific for human CYP3A4, CYP3A7, HNF-1α, HNF-1β, HNF-4α and β-actin (Begum et al.). The primers were selected such as to anneal to one upstream and one downstream exon with an intervening intron. Amplification conditions were as follows: an initial denaturation at 95° C. for 5 min was followed by cycles of denaturation at 95° C. for 45 s, annealing at 50-65° C. (Table 2) for 15 s, extension for 1 min at 72° C., and a final polymerization at 72° C. for 10 min. PCR products were analyzed by gel electrophoresis in 2% agarose gels, stained with, GelRed (Biotium, Inc, Hayward, Calif., USA) visualized on a UV transilluminator (Hoefer Inc., San Francisco, Calif., USA) and documented using the ULTima AQ software (Hoefer Inc.). cDNA from primary adult hepatocytes was used as positive controls (14).

Flow Cytometry.

The cells were washed with BSA/PBS by centrifugation at 1,500 RPM for 10 minutes and the supernatant was poured off BD Cytofix/Cytoperm Kit (BD Biosciences, San Jose, Calif. USA) was added according to the manufacturer to the pellet. The cells were washed again with BSA/PBS and the following primary antibodies were added: mouse anti-human CD 326 (Morphosys AbD GmbH, Düsseldorf, Germany)( ) Dlk-1 (Santa Cruz Biotechnology, Santa Cruz, Calif., USA), CD 144 (BD Biosciences San Jose, Calif. USA), CD 133 ((Miltenyi Biotec GmbH, Gladbach, Germany, CD34 CD45, CD90 (all from BD Pharmingen, San Jose, Calif. USA), CD 271 (BD Biosciences, San Jose, Calif. USA), CK8 (Santa Cruz Biotechnology, Santa Cruz, Calif., USA), and albumin (Bethyl Laboratories, Inc., TX, USA). After incubating the cells at 4° C. for 30 minutes, they were washed in BSA/PBS again. Secondary antibody Alexa Fluor 488-conjugated rabbit anti-mouse IgG, (Invitrogen Ltd. Eugene, Oreg., USA) was added and incubated for 30 minutes at 4° C. The cells were washed and resuspended in 200 μl of PBS for flow cytometric analysis. Cells were analysed on a FACSsorter (Becton Dickinson, USA). Fluorescent signals from 10,000 cells were counted and the percentage of positively stained cells was recorded.

Transplantation Studies

The animal care and ethics committee at Sahlgrenska university hospital in Gothenburg, Sweden approved of the animal protocols. Liver injury was induced in nude C57BL/6 mice (n=7) by administration of retrorsine (Sigma Chemicals Co., Stockholm, Sweden) as described by us earlier (14). SV40 LT HFL cells were transplanted into the spleens of these animals. Animals were anaesthetized under isofluorane and 1×10⁶ cells in 200 μl of DMEM medium were injected into the spleen over approximately 10-15s. Four mice were sham-transplanted with just DMEM medium. After securing hemostasis, the abdominal incision was closed and the animals were monitored closely until recovery.

Preparation of Livers and Analysis of Fluorescence in Cryosections

Mice were sacrificed 4 weeks after transplantation and their livers, spleens and lungs were excised. The liver tissue was shock frozen in liquid nitrogen for fluorescence and immunohistochemical analyses. Cryosections, 5 μm in thickness, were air dried and fixed with cold 30% acetone in methanol for 10 min and further analysed by immunohistochemistry.

Immunohistochemistry

The tissue slides were incubated overnight at 4° C. with antibodies specific for human α-fetoproein diluted 1:200 in PBS, human nuclear antigen diluted 1:100 (Millipore, Stockholm, Sweden), human c-Met diluted 1:100 (RDI, Concord, Mass., USA), human CK8 diluted 1:200, human CK18 diluted 1:200, human CK19 diluted 1:200, human hepatocyte-specific antigen diluted 1:100 (Santa Cruz Biotechnology) and anti-p53. The rest of the procedure is described by us in detail elsewhere (14). 3,3′-diaminobenzidine tetrahydrochloride (DAB; Immunkemi AB) was used as a chromogen. Tissues were counterstained by hematoxylin. Negative controls were processed by replacing the primary antibody with diluent only, followed by secondary antibody. As a positive control, sections of liver cancerous tissue or normal liver tissue were immunostained using the different antibodies. Immunofluorescent staining for detection of human albumin was performed using FITC-conjugated goat anti-human albumin antibody diluted 1:200. Sections were counterstained with DAPI as before, mounted with aqueous mounting medium (Vector Laboratories, Burlingame, Calif., USA), and examined under the fluorescence microscope.

### Example 2

### Generation and Growth of SV40 LT Antigen-Immortalized Human Fetal Liver Cells

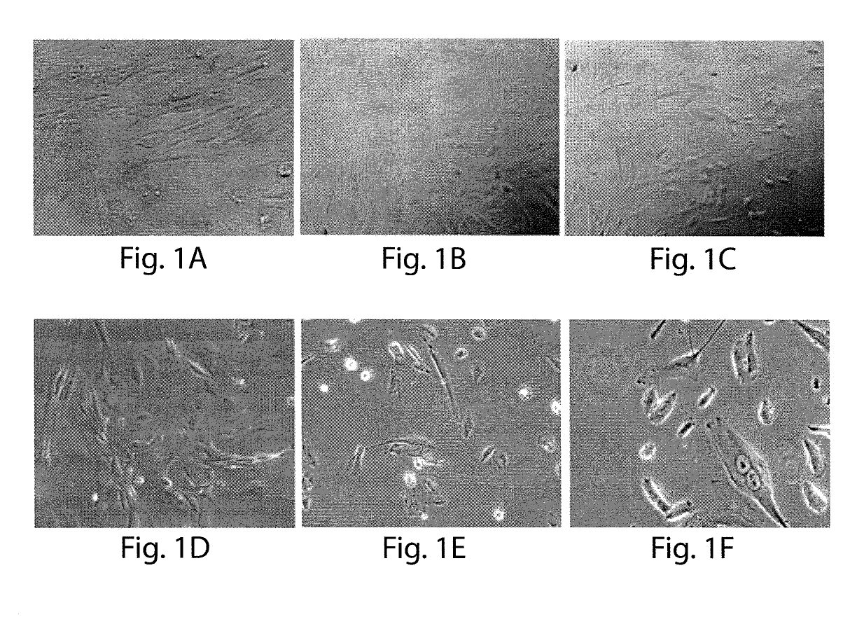

Colonies of cells grew in the selection marker puromycin and these cells were passaged onto new culture flasks and handled as separated bulk clones. So far, we have investigated cells in one of the bulk clones. To investigate whether immortalized cells retain the morphologic characteristics of primary liver cells, the cells were examined by phase contrast microscopy (FIG. 1). Immortalized hepatocytes grew in clusters of closely apposed cells of typical morphology including large size, poly- or hexagonal shape and with more than one nucleus (FIG. 1A-F).

Following plating of SV40LT-HLF cells, there is a lag phase of 0-48 hrs before the cells start to grow exponentially. The population doubling time was 30.8 for cells that have been cultured for 2.5 months. The culture reached confluency after 3 days and the cell numbers had dropped following another 3 days in culture. The relative quick decrease in cell numbers when the culture reach confluency indicates that the stable transfected cells in the SV40LT line inhibit growth via contact inhibition as normal cells do.

### Example 3

### Phenotypic Characterization of SV40 Large T Antigen-Immortalized Human Fetal Liver Cells

Expression of Hepatic Markers.

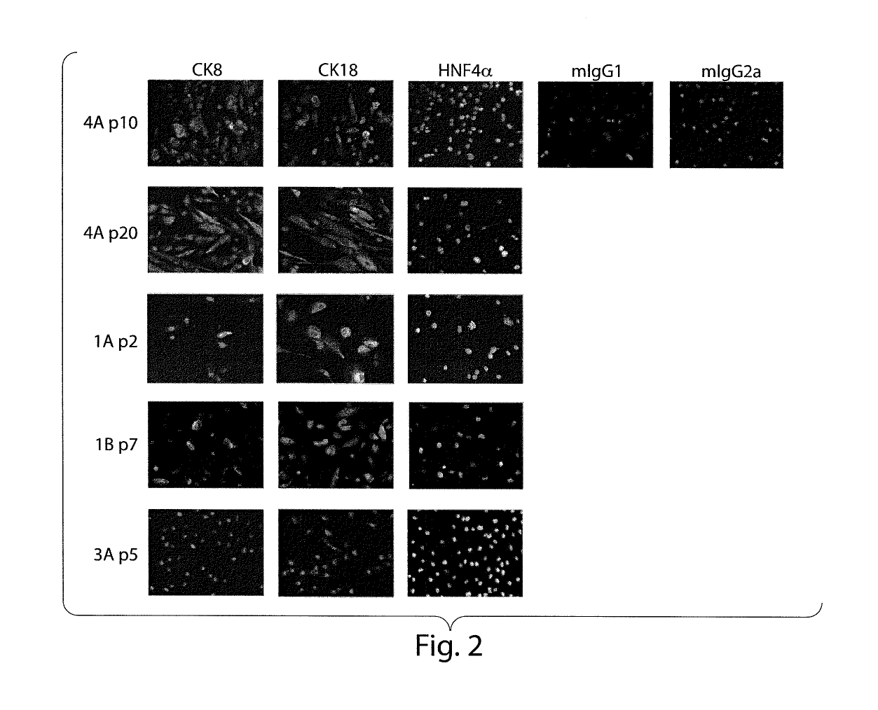

Detection by immunocytochemistry of human hepatocyte-specific proteins in transfected fetal hepatocytes revealed expression of the hepatocyte marker proteins, CK 8 and CK 18, as clear cytoplasmic filamentous structures both in early (p2-p10) as well as late (p20) passage cells (FIG. 2, column 1 and 2). In general, the staining was distributed equally in almost all the cells of both early and late passages. However, while cells from the majority of the bulk clones expressed both CK8 and CK18, no positive staining for CK8 and only faint staining for CK18 were detected in cells of bulk clone 3A (FIG. 2 column 1 and 2).

Furthermore, the transcription factor HNF-4α (FIG. 2, column 3) was expressed in the SV40 LT-HFL cells. It was detected in the nuclei of nearly all the cells in early passage cells. Also in this case, this marker was expressed in passage 20 (FIG. 2, column 3, panel 2). No positive staining was observed in any of the isotype controls (FIG. 2, column 4 and 5).

Expression of the SV40 LT Antigen and Tumor-Associated Markers.

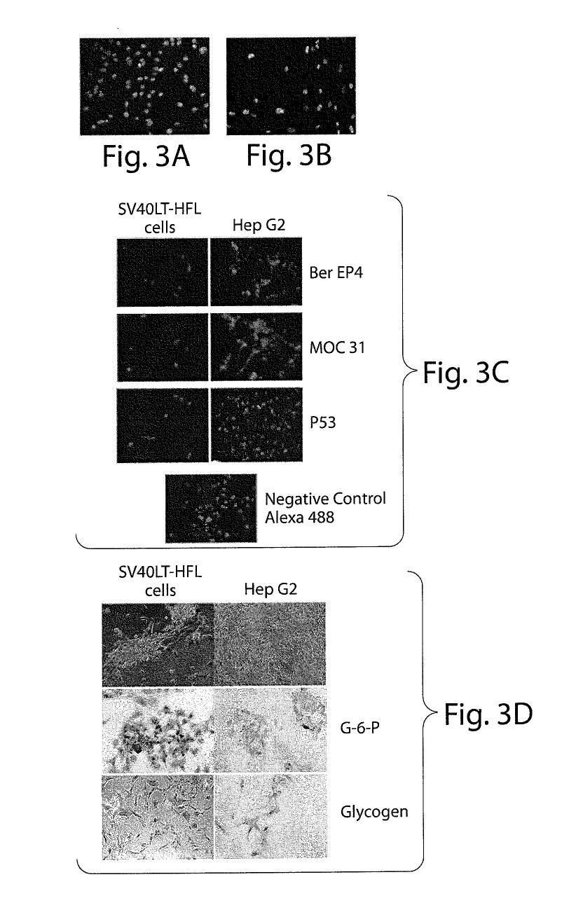

As determined by immunocytochemistry, virtually all SV40LT-HFL cells that survived continuous puromycin selection expressed SV40 large T antigen. FIG. 3 shows a representative staining of SV40LT in cells of bulk clone 4A in passage 10 and 20 (FIGS. 3A and B, respectively). The nuclear staining appeared to be of similar intensity (FIGS. 3A and B). The SV40 large T antigen expression in SV40 LT-HFL cells was consistent during cell culture suggesting stable integration into the host genome of the plasmid encoding SV40 LT antigen. In addition, the transfected cells did not stain positively for the cancer-associated markers p53, MOC-31 or Ber EP4, while the cancer cell line HepG2 cell line strongly expressed MOC-31 and Ber EP4, but not p53 (FIG. 3C). Furthermore, using immunocytochemistry and histochemical analysis, we found that the transfected cells expressed the liver specific marker glucose-6-phosphatase (G-6-P) and stored glycogen (FIG. 3D).

Expression of Stem Cell Markers.

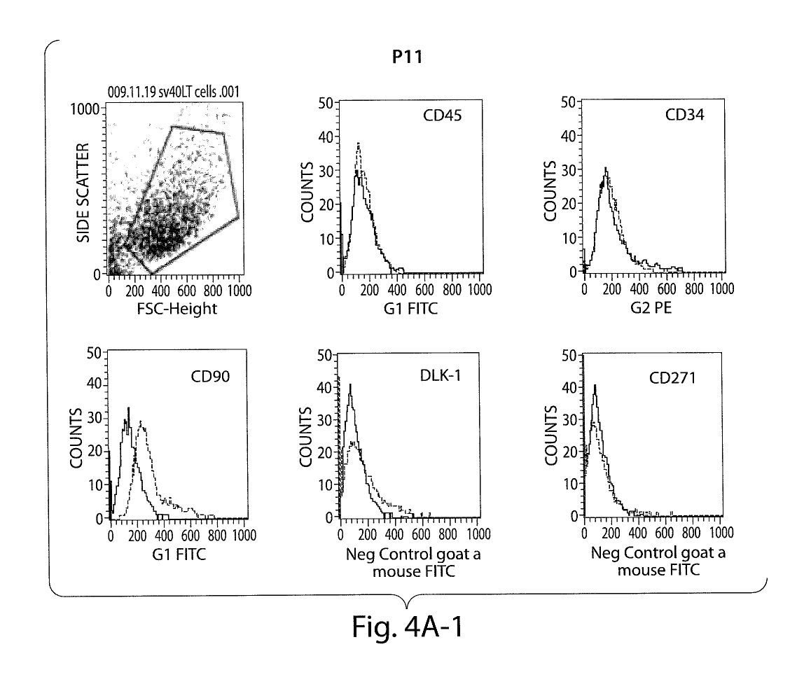

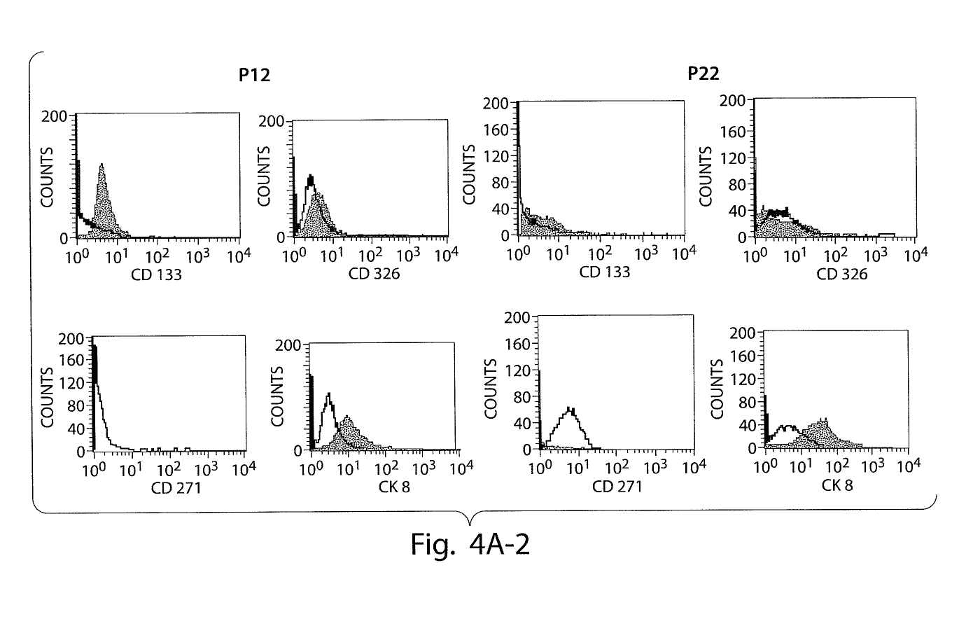

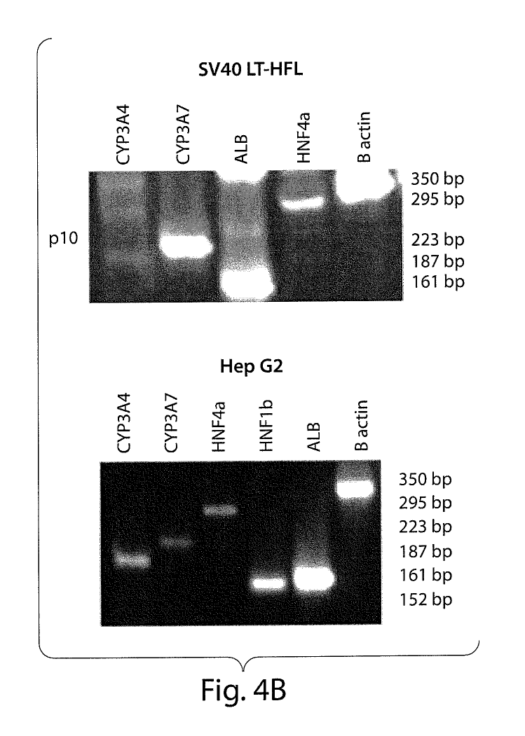

Hepatic stem markers such as EPCAM, Dlk-1, CD90 and CD133 was detected in early passages of SV40LT-HFL cells (p11-12), but the expression of these markers were found to be decreased or absent the in late passages (p22) using flow cytometry (FIG. 4A). We also confirmed expression of CK 8 in the cells, which were strongly expressed in both early and late passages (FIG. 4A). Interestingly, the cells did not express the mesenchymal stem cell marker, CD271 (FIG. 4A). Furthermore, the cells did not express the hematopoietic stem cell marker, CD34, or the leukocyte marker, CD45.

mRNA Detection by Reverse Transcriptase—PCR

To determine whether the immortalized cells retained liver-specific protein expression, markers of hepatocytes were also analyzed by RT-PCR. The results indicated that the immortalized HFL cells (bulk clone 4A) were positive for albumin, CYP3A4/7, and HNF-4α (FIG. 4B). There was a pronounced difference in the level of gene expression of CYP3A7 as compared to that of CYP3A4. Housekeeping gene was β-actin (FIG. 4B). Hep G2 cells were used as control cells (FIG. 4C).

### Example 4

### Human Fetal Liver Cells Immortalized by SV40 Large T Antigen Expression Successfully Differentiate into Functionally Mature Hepatocytes and Cholangiocytes in the Livers of Retrorsine-Treated Mice

To test whether SV40 LT-HFL cells have the potential to differentiate and be functional in vivo, we transplanted bulk clone 4A cells into mice that were first treated with retrorsine to induce liver injury and then partially hepatectomized. All mice receiving the cells survived at four weeks (n=7) after cell transplantation.

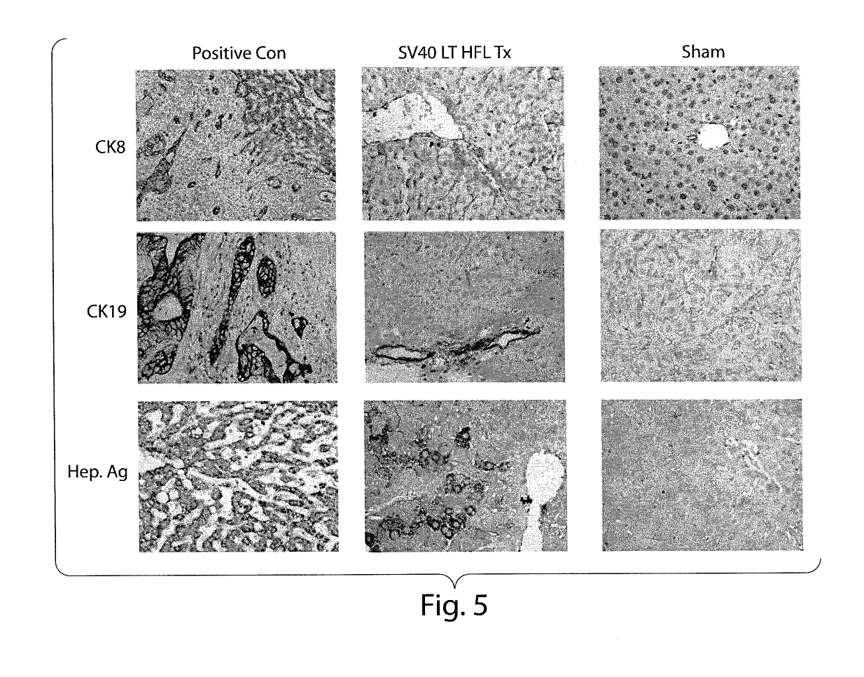

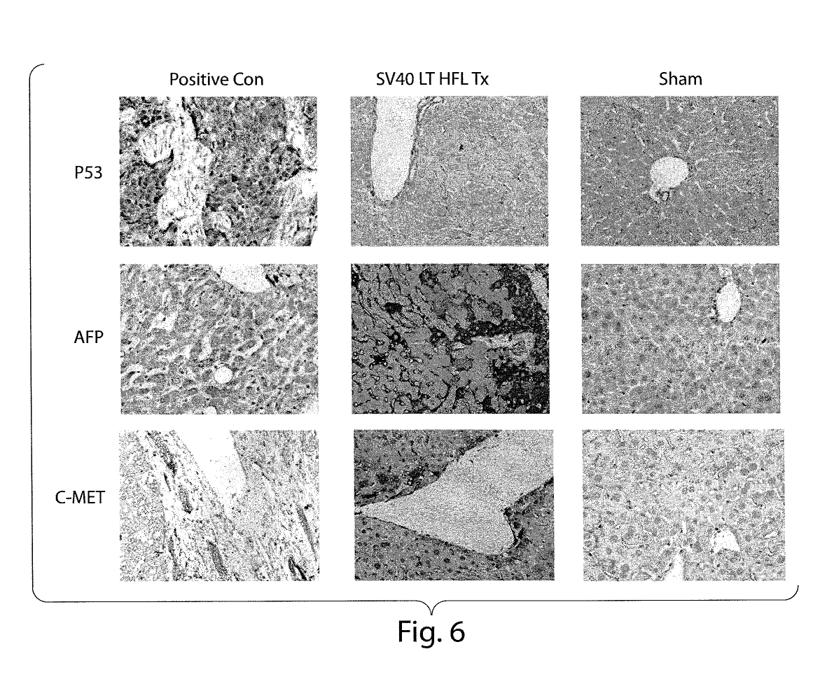

The transplanted cells differentiated into hepatocytes (FIGS. 5 and 6), and formed bile ducts (FIG. 5) at four weeks after transplantation. We also observed clear areas of bile ducts repopulated by human progenitors. These cells stained positively with an antibody specific for human CK19 (FIG. 5). We found several human cells expressing the hepatocyte-specific antigen (FIG. 5) in all of the seven transplanted mice. In addition, several clusters of cells expressing human CK8, human AFP and human c-Met were also found in the transplanted animals. Furthermore, the transplanted cells did not express the tumor suppressor marker p53 (FIG. 6). As a note of interest no tumor formations were observed in any of the mice analyzed and the architecture of the liver was found to be normal.

### REFERENCES

1. Barbich M, Lorenti A, Hidalgo A, Ielpi M, de Santibáñez, de Santibáñez E, Morales V, Marin M C, Callero M F, Argibay P F. Culture and characterization of human hepatocytes obtained after graft reduction for liver transplantation: a reliable source of cells for a bioartificial liver. Artif Organs. 2004 July; 28(7):676-82.
2. Fox I J, Roy-Chowdhury J: Hepatocyte transplantation. J Hepatol 40: 878-886, 2004.
3. Kobayashi N, Fujiwara T, Westerman Kans., Inoue Y, Sakaguchi M, Noguchi H, Miyazaki M, Cai J, Tanaka N, Fox I J, Leboulch P., Prevention of acute liver failure in rats with reversibly immortalized human hepatocytes. Science 287: 1258-1262, 2000.
4. May P, May E. Twenty years of p53 research: structural and functional aspects of the p53 protein. Oncogene. 1999 Dec. 13; 18(53):7621-36.
5. Ali S H, DeCaprio J A. Cellular transformation by SV40 large T antigen: interaction with host proteins. Semin Cancer Biol. 2001 February; 11(1):15-23.
6. Pipas J M, Levine A J. Role of T antigen interactions with p53 in tumorigenesis. Semin Cancer Biol. 2001 February; 11(1):23-30.
7. Shay J W, Wright W E, Werbin H. Defining the molecular mechanisms of human cell immortalization. Biochim Biophys Acta. 1991 Apr. 16; 1072(1):1-7. Review.
8. Wadhwa R, Kaul S C, Mitsui Y. Cellular mortality and immortalization: a complex interplay of multiple gene functions. Prog Mol Subcell Biol. 2000; 24:191-204. Review.
9. Toouli C D, Huschtscha L I, Neumann A A, Noble J R, Colgin L M, Hukku B, Reddel R R. Comparison of human mammary epithelial cells immortalized by simian virus 40 T-Antigen or by the telomerase catalytic subunit. Oncogene. 2002 Jan. 3; 21(1):128-39.)
10. Kobayashi N, Fujiwara T, Westerman K A, et al: Prevention of acute liver failure in rats with reversibly immortalized human hepatocytes. Science 287: 1258-1262, 2000.
11. Kobayashi N, Miyazaki M, Fukaya K, Inoue Y, Sakaguchi M, Noguchi H, Tanaka N, Namba M. Establishment of a highly differentiated immortalized human hepatocyte cell line as a source of hepatic function in the bioartificial liver. Transplant Proc. 2000 March; 32(2):237-41
12. Kobayashi N, Noguchi H, Fujiwara T, Westerman Kans., Leboulch P, Tanaka N: Establishment of a highly differentiated immortalized adult human hepatocyte cell line by retroviral gene transfer. Transplant Proc 32: 2368-2369, 2000.
13. Andres D, Diez-Fernandez C, Zaragoza A, Alvarez A, Cascales M: Induction of cell proliferation by cyclosporine A in primary cultures of rat hepatocytes. Biochem Pharmacol 61: 427-435, 2001.
14. Begum S, Joshi M, Ek M, Holgersson J, Kleman M I and Sumitran-Holgersson S: Characterization and engraftment of long-term serum-free human fetal liver cell cultures. Cytotherapy 2009. In press.
15. Rutenberg, A. M. Histochemical and ultrastructural demonstration of γ-glutamyl transpeptidase activity. J. Histochem. Cytochem. 17, 517-526, 1969.
16. Ott, M., Rajvanshi, P., Sokhi, R. P., Alpini, G., Aragona, E., Dabeva, M., Shafritz, D. A. and Gupta S. Differentiation-specific regulation of transgene expression in a diploid epithelial cell line derived from the normal F344 rat liver. J. Pathol. 187, 365-373, 1999.
17. Toouli C D, Huschtscha L I, Neumann A A, Noble J R, Colgin L M, Hukku B, Reddel R R. Comparison of human mammary epithelial cells immortalized by simian virus 40 T-Antigen or by the telomerase catalytic subunit. Oncogene. 2002 Jan. 3; 21(1):128-39.
18. U Latza, G Niedobitek, R Schwarting, H Nekarda, H Stein. Ber-EP4: new monoclonal antibody which distinguishes epithelia from mesothelial. Journal of Clinical Pathology 1990; 43:213-219.19. Ordóñez NG. Value of the MOC-31 monoclonal antibody in differentiating epithelial pleural mesothelioma from lung adenocarcinoma. Hum Pathol. 1998 February; 29(2):166-9.
20. Woodworth C D, Kreider J W, Mengel L, Miller T, Meng Y L, Isom H C. Tumorigenicity of simian virus 40-hepatocyte cell lines: effect of in vitro and in vivo passage on expression of liver-specific genes and oncogenes. Mol Cell Biol. 1988 October; 8(10):4492-501.
21. Kim B H, Sung S R, Choi E H, Kim Y I, Kim K J, Dong S H, Kim H J, Chang Y W, Lee J I, Chang R. Dedifferentiation of conditionally immortalized hepatocytes with long-term in vitro passage. Exp Mol. Med. 2000 Mar. 31; 32(1):29-37.22.
22. Zhang X Y, Guckian M, Nasiri N, Lovell P A, Dalgleish A G, Barton D P. Normal and SV40 transfected human peritoneal mesothelial cells produce IL-6 and IL-8: implication for gynaecological disease. Clin Exp Immunol. 2002 August; 129(2):288-96.
23. Shiojiri N. The origin of intrahepatic bile duct cells in the mouse. J Embryol Exp Morphol. 1984 February; 79:25-39.
24. Suzuki A, Zheng Y W, Kaneko S, Onodera M, Fukao K, Nakauchi H, Taniguchi H. Clonal identification and characterization of self-renewing pluripotent stem cells in the developing liver. J Cell Biol. 2002 Jan. 7; 156(1):173-84. Epub 2002 Jan. 7.
25. Schmelzer E, Zhang L, Bruce A, Wauthier E, Ludlow J, Yao H L, Moss N, Melhem A, McClelland R, Turner W, Kulik M, Sherwood S, Tallheden T, Cheng N, Furth M E, Reid L M. Human hepatic stem cells from fetal and postnatal donors. J Exp Med. 2007 Aug. 6; 204(8):1973-87. Epub 2007 Jul. 30.
26. Grompe M. The role of bone marrow stem cells in liver regeneration. Semin Liver Dis. 2003 November; 23(4):363-72.
27. Kubota H, Reid L M. Clonogenic hepatoblasts, common precursors for hepatocytic and biliary lineages, are lacking classical major histocompatibility complex class I antigen. Proc Natl Acad Sci USA. 2000 Oct. 24; 97(22):12132-7.

What is claimed is:

1. An immortalized human fetal liver cell line containing actively expressing SV40 LT genes wherein the said immortalized cell line maintains the phenotypic properties of human fetal liver cells and is capable of differentiating in vivo into a hepatocyte, or a cholangiocyte.

2. An immortalized in vitro cell culture comprising cells derived from fetal liver tissue of a human, wherein said cells in the culture
a. express albumin, cytokeratin 8 (CK8), cytokeratin 18 (CK18), cytokeratin 19 (CK19), cytochrome P450 3A4/7 isoenzyme, HNF4α, glucose-6-phosphatase
b. do not express cancer associated markers
c. are CD271⁻, CD34⁻ and CD35⁻
d. are capable of proliferating in a culture; and
e. are capable of differentiating in vivo into a hepatocyte, or a cholangiocyte.

3. The cell culture of claim 2, wherein said cells store glycogen.

4. The cell culture of claim 2, wherein said cancer associated markers are p-53, MOC-31 or Ber EP4.

5. The cell culture of claim 2, wherein when said cells do not form tumors in vivo.

6. A method of screening for compounds which effect proliferation, differentiation or survival of liver cells comprising
a. providing the cell line of claim 1 or the cell culture of claim 2;
b. contacting said cell line or culture with a test compound; and
c. determining if said compound has an effect on proliferation, differentiation or survival of said cell line or cell culture.

7. An in vitro method for determining a metabolite of a test compound comprising
a. providing the cell line of claim 1 or the cell culture of claim 2;
b. contacting said cell line or culture with the test compound; and
c. identifying a metabolite derived from the test compound after incubation with said cell line or culture.

8. An in vitro method of determining the anti-viral activity of a test compound comprising:
a. providing the cell line of claim 1 or the cell culture of claim 2;
b. contacting said cell line or culture with a virus and a test compound; and
c. comparing the survival rate of said cell line or culture with a control cell culture;
wherein in an increase in survival rate of said cell line or culture compared to said control culture indicates anti-viral activity of said test compound

9. An in vitro method of determining the infectivity of a virus comprising:
a. providing the cell line of claim 1 or the cell culture of claim 2;
b. contacting said culture with a virus; and
c. determining if said virus has an effect in proliferation or survival of said cell line or culture.
